(19) United States
(12) Patent Application Publication
(10) Pub. No.: US 2026/0294517 A1
(43) Pub. Date: Oct. 1, 2026

(54) TECHNOLOGIES FOR MAINTAINING ELECTRICAL CONNECTIVITY IN ROTATING COMPONENTS OF ENERGY-BASED SURGICAL INSTRUMENTS

(71) Applicant: Cilag GmbH International, Zug (CH)

(72) Inventors: Tyler N. Brehm, Cincinnati, OH (US); Jacob Gee, Cincinnati, OH (US); Matthew Bruchas, Cincinnati, OH (US)

(21) Appl. No.: 19/410,634

(22) Filed: Dec. 5, 2025

Related U.S. Application Data
(60) Provisional application No. 63/780,379, filed on Mar. 30, 2025.

(51) Int. Cl. A61B 18/14; A61B 18/00

(52) U.S. Cl. CPC A61B 18/1445; A61B 2018/00077; A61B 2018/00178; A61B 2018/00184; A61B 2018/0091; A61B 2018/00994; A61B 2018/1452

(57) ABSTRACT

A surgical instrument includes an elongated shaft assembly, a trigger assembly, and an electrical rotating sleeve. The elongated shaft assembly has an outer tubular sheath and an end effector. The end effector has a jaw assembly movable between an open state and a closed state, and the jaw assembly includes at least one electrode. The trigger assembly is operable to move the jaw assembly of the end effector between the open state and the closed state. The electrical rotating sleeve delivers bipolar energy to the at least one electrode of the jaw assembly through the outer tubular sheath.

This application claims priority to U.S. Provisional Patent Application Serial No. 63/780,379, which was filed on Mar. 30, 2025, the entirety of which is hereby incorporated by reference.

### TECHNICAL FIELD

The present disclosure relates generally to energy-based surgical instruments and, more particularly, to surgical instruments and associated methods for controlling rotating electrical contacts on surgical devices to deliver energy to tissue.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient’s tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient’s tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100 kHz to about 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities, which increases the surgical options provided by the surgical instrument to the surgeon.

Some surgical instruments may include one or more rotating or otherwise movable parts. In such surgical instruments, an electrical connection may be required to be maintained across or through such rotating movable part. However, a rotating or movable electrical connections can be difficult to design to achieve a robust connection and reliable assembly. For example, under certain circumstances, typical rotating or movable electrical connections or contacts may be prone to failure, which can result in open circuits and failure of energy delivery to the tissue.

### SUMMARY

According to an aspect of the present disclosure, a surgical instrument may include an elongated shaft assembly having an outer tubular sheath and an end effector positioned at a distal end of the outer tubular sheath. The end effector may have a jaw assembly movable between an open state and a closed state. The jaw assembly may include at least one electrode and an ultrasonic blade. The surgical instrument may also include a trigger assembly operable to move the jaw assembly of the end effector between the open state and the closed state and a washer in contact with the trigger assembly. The surgical instrument may further include an electrical rotating sleeve for delivering bipolar energy to the at least one electrode of the jaw assembly via the outer tubular sheath. The washer may be received on the electrical rotating sleeve and a proximal end of the outer tubular sheath may be received within the electrical rotating sleeve. An electrical connection may be established between the trigger assembly, the washer, the electrical rotating sleeve, and the outer tubular sheath to deliver the bipolar energy to the at least one electrode.

In some embodiments, the electrical rotating sleeve includes a collar having an internal wall that defines a bore in which the outer tubular sheath is received and a cylindrical overmold coupled to the collar. In such embodiments, the cylindrical overmold may include a plurality of external threads formed on an outer surface thereof. Additionally, in such embodiments, the washer may extend around the collar of the electrical rotating sleeve to provide electrical contact between the trigger assembly and the outer tubular sheath via the collar.

Additionally or alternatively, the trigger assembly may include a primary trigger and a yoke configured to move along a yoke axis in response to operation of the primary trigger to move the jaw assembly of the end effector between the open state and the closed state. In such embodiments, the washer may be in contact with the yoke of the trigger assembly. In some embodiments, the yoke of the trigger assembly and the collar of the electrical rotating sleeve may be formed of an electrically conductive material. Additionally or alternatively, the yoke may include a support arm and a contact strip, and the contact strip may extend along the support arm and is in electrical contact with the washer. Additionally, in some embodiments, the contact strip of the yoke and the collar of the electrical rotating sleeve may be formed of electrically conductive materials, and the support arm of the yoke may be formed of non-electrically conductive materials.

In some embodiments, the electrical rotating sleeve may further include an electrical ring disposed between the collar and the cylindrical overmold. The electrical ring may have a base and a plurality of fingers extending distally away from the base toward the collar. The plurality of fingers may cooperate to define a finger channel between each pair of adjacent fingers of the plurality of fingers. The washer may extend around and be in electrical contact with the electrical ring of the electrical rotating sleeve to provide electrical contact between the trigger assembly and the outer tubular sheath via the electrical ring. In such embodiments, the collar of the electrical rotating sleeve may include a band, a flange extending outwardly away from the band at a distal end thereof, and a plurality of tabs extending proximally away from a proximal end of the band toward the electrical ring. The plurality of tabs may cooperate to define a tab channel between each pair of adjacent tabs of the plurality of tabs.

In some embodiments, each of the plurality of fingers of the electrical ring may be received in a corresponding tab channel of the plurality of tab channels. Additionally, each of the plurality of tabs of the band may be received in a corresponding finger channel of the plurality of finger channels. Furthermore, in some embodiments, the cylindrical overmold may include a body and a plurality of extensions extending distally away from the body into the plurality of finger channels of the electrical ring and into contact with the plurality of tabs of the collar.

According to another aspect of the present disclosure, a surgical instrument may include an elongated shaft assembly, a trigger assembly, and an electrical rotating sleeve. The elongated shaft assembly may have an outer tubular sheath and an end effector positioned at a distal end of the outer tubular sheath. The end effector may have a jaw assembly movable between an open state and a closed state, wherein the jaw assembly includes at least one electrode. The trigger assembly may be operable to move the jaw assembly of the end effector between the open state and the closed state. The electrical rotating sleeve may be configured for delivering bipolar energy to the at least one electrode of the jaw assembly through the outer tubular sheath. The electrical rotating sleeve may be configured to receive a proximal end of the outer tubular sheath therein to rotate therewith. The bipolar energy may be directed through the trigger assembly, to the electrical rotating sleeve, through the outer tubular sheath, and to the at least one electrode.

In some embodiments, the electrical rotating sleeve may include a collar having an internal wall that defines a bore in which the outer tubular sheath is received and a cylindrical overmold coupled to the collar. The cylindrical overmold may include a plurality of external threads formed on an outer surface thereof. In such embodiments, the surgical instrument may further include a washer that extends around the collar of the electrical rotating sleeve to provide electrical contact between the trigger assembly and the outer tubular sheath via the collar.

In some embodiments, the electrical rotating sleeve may further include an electrical ring disposed between the collar and the cylindrical overmold. The electrical ring may have a base and a plurality of fingers extending distally away from the base toward the collar. The surgical instrument may further include a washer that extends around and is in electrical contact with the electrical ring to provide electrical contact between the trigger assembly and the outer tubular sheath via the electrical ring. In such embodiments, the collar may include a band, a flange extending outwardly away from the band at a distal end thereof, and a plurality of tabs extending proximally away from a proximal end of the band toward the electrical ring, and wherein each of the plurality of fingers of the electrical ring is received between adjacent tabs of the plurality of tabs of the collar. Additionally, in some embodiments, the cylindrical overmold may include a body and a plurality of extensions extending distally away from the body. Each of the plurality of extensions of the cylindrical overmold may be received between adjacent fingers of the plurality of fingers of the electrical ring. Additionally, each of the plurality of extensions extends over a corresponding tab of the plurality of tabs of the collar.

According to yet a further aspect of the present disclosure, a method may include positioning a proximal end of an outer tubular sheath of an elongated shaft assembly in an electrical rotating sleeve to provide electrical contact between the outer tubular sheath and the electrical rotating sleeve, positioning a washer around the electrical rotating sleeve to provide electrical contact between the washer and the electrical rotating sleeve, contacting the washer with a yoke of a trigger assembly to provide electrical contact between the yoke and the washer, and directing bipolar energy through the yoke, to the washer, to the electrical rotating sleeve, through the outer tubular sheath, and to at least one electrode of a jaw assembly included in the elongated shaft assembly.

In some embodiments, the electrical rotating sleeve mayinclude a collar, a cylindrical overmold, and an electrical ring disposed between the collar and the cylindrical overmold. The collar and the cylindrical overmold may be formed of non-electrically conductive materials. In such embodiments, positioning the proximal end of the outer tubular sheath of the elongated shaft assembly in the electrical rotating sleeve to provide electrical contact between the outer tubular sheath and the electrical rotating sleeve may include contacting the electrical ring of the electrical rotating sleeve with the proximal end of the outer tubular sheath. Additionally or alternatively, positioning the washer around the electrical rotating sleeve to provide electrical contact between the washer and the electrical rotating sleeve may include contacting the electrical ring of the electrical rotating sleeve with the washer.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 in a closed state;

FIG. 5A is a perspective view of another embodiment of an end effector of the surgical instrument of FIG. 2 including an electrode on a lower jaw clamp of the jaw assembly;

FIG. 5B is a perspective view of another embodiment of an end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a perspective view of an embodiment of an energy-based surgical instrument of the system of FIG. 1 having a portion of a housing removed to illustrate internal components;

FIG. 9 is a block diagram of an electrical connection that is established through the energy-based surgical instrument of FIG. 8;

FIG. 10 is an enlarged view of a portion of the internal components of the energy-based surgical instrument of FIG. 8;

FIG. 11 is a perspective view of an electrical rotating sleeve of the surgical instrument of FIG. 10;

FIG. 12 is a block diagram of another embodiment of an electrical connection that is established through another embodiment of an energy-based surgical instrument of the system of FIG. 1;

FIG. 13 is an enlarged view of a portion of internal components of the energy-based surgical instrument having the electrical connection of FIG. 12;

FIG. 14 is a perspective view of a contact strip configured to be coupled to a yoke of the surgical instrument of FIG. 13;

FIG. 15 is a perspective view of the yoke of FIG. 13 having the contact strip coupled thereto;

FIG. 16 is a block diagram of another embodiment of an electrical connection that is established through another embodiment of an energy-based surgical instrument of the system of FIG. 1;

FIG. 17 is an enlarged view of a portion of the internal components of the energy-based surgical having the electrical connection of FIG. 16;

FIG. 18 is a perspective view of an electrical rotating sleeve of the surgical instrument of FIG. 17;

FIG. 19 is an exploded view of the electrical rotating sleeve of FIG. 18;

FIG. 20 is a side cross-sectional view of the electrical rotating sleeve of FIGS. 18 and 19; and

FIG. 21 is a side cross-sectional view of the electrical rotating sleeve of FIGS. 18-20 disposed around an outer tubular sheath of the surgical instrument of FIG. 17.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient’s natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

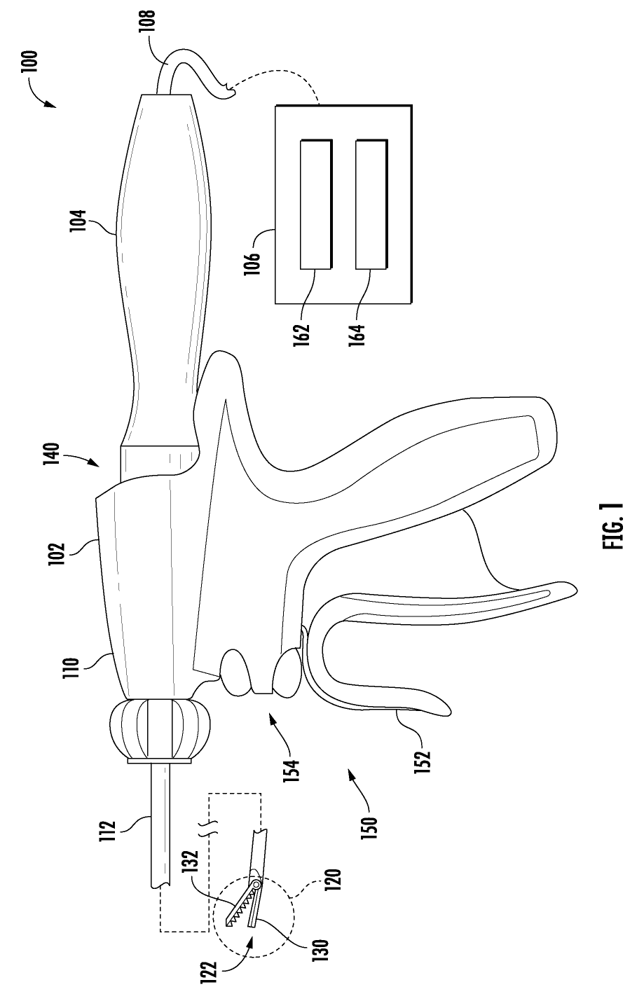

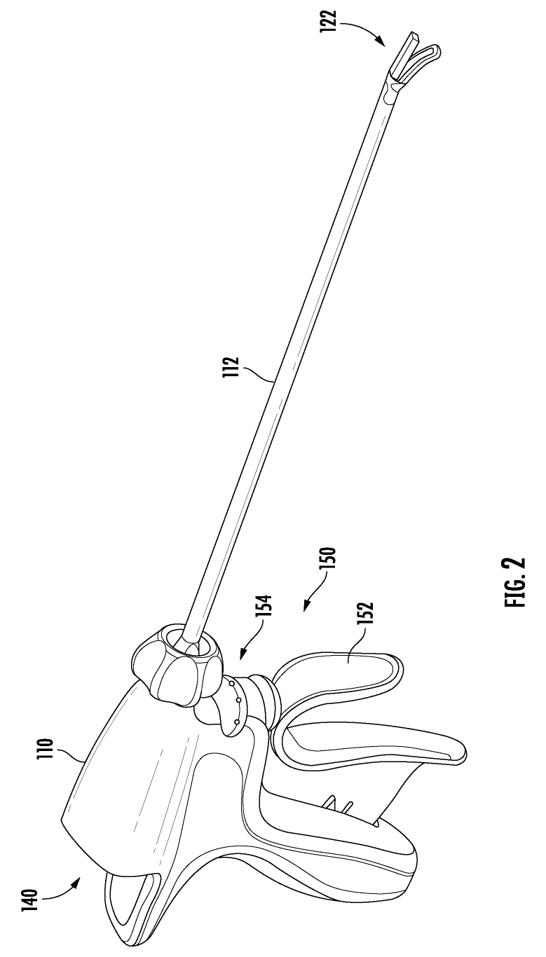

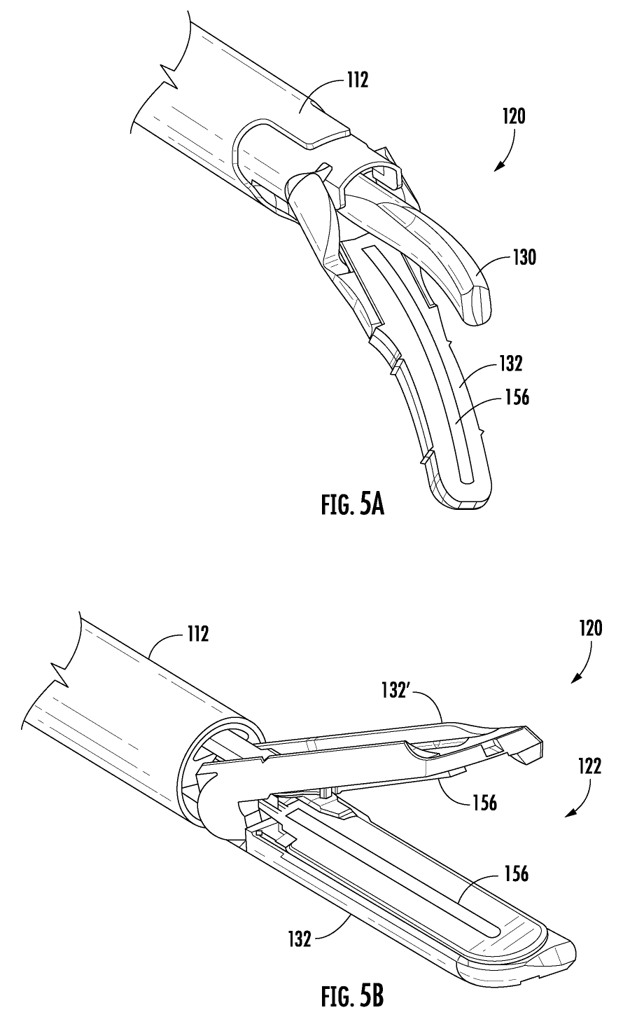

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

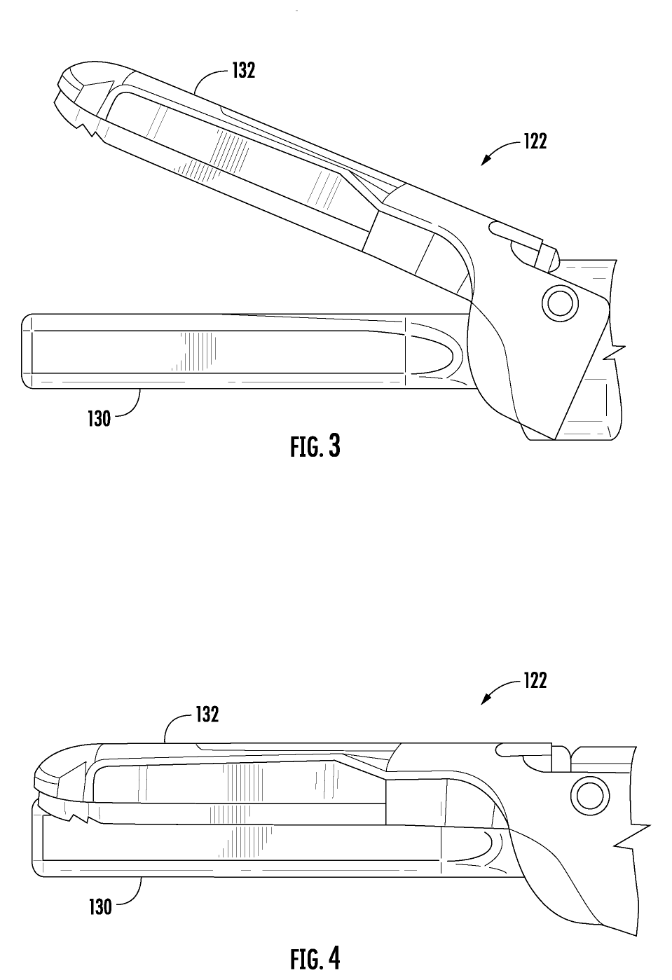

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 156 incorporated into the jaw clamp 132 as shown in FIG. 5A. Although the illustrative end effector 120 includes only a single electrode 156 in the embodiment of FIG. 5A, it should be appreciated that the end effector 120 may include additional electrodes 156 in other embodiments (e.g., multiple pads of electrodes 156). The electrode(s) 156 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 156 embodying the other active or return electrode. Alternatively, other active or return electrodes may be incorporated on the ultrasonic blade 130 or in another part of the jaw assembly 122 of the end effector 120. In mono-polar implementation, the RF electrode(s) 156 may be embodied as an active electrode, and a return electrode may be attached to a portion of the patient’s body.

In those embodiments in which the surgical instrument 102 includes only an electro-surgical/RF mode, the jaw assembly 122 of the end effector 120 includes a jaw clamp 132′ in place of the ultrasonic blade 130 as shown in FIG. 5B. In such embodiments, the electrode 156 may be attached to or otherwise incorporated into each jaw clamp 132, 132′ and be embodied as an active or a return electrode to facilitate the application of RF energy to tissue captured between the jaw clamps 132, 132′. In such embodiments, the surgical instrument 102 may include a knife incorporated into the elongated shaft assembly 112 that is configured to eject outwardly to cut the patient’s tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kHz to about 1 MHz. The generated RF energy is supplied to the patient’s tissue via the electrodes 156 of the end effector 120 as described above in regard to FIGS. 5A and 5B. In some embodiments, the electro-surgical generator module 164 may also be configured to selectively provide the RF energy at sub-therapeutic levels to perform various electrical measurements of the patient’s tissue. For example, the electro-surgical generator module 164 may be configured to measure an impedance of the patient’s tissue using the electrodes 156 and a suitable RF energy level.

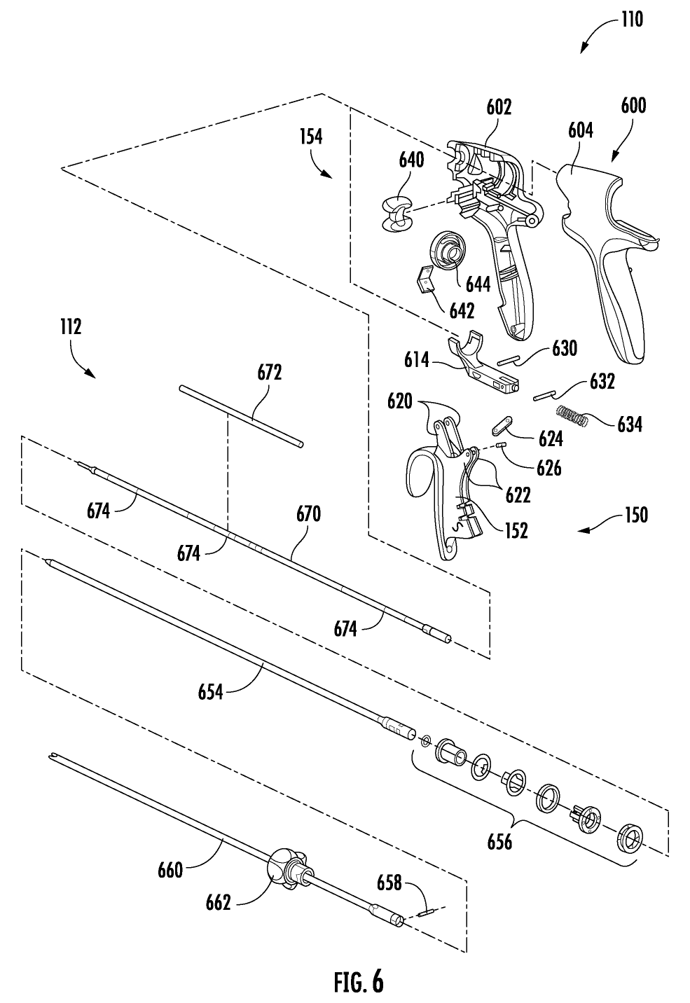

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half housings 602, 604 are configured to mate with each other to form the housing 600. To facilitate such mating, each of the half housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 614, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 614. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 614 and retained therein by a second yoke pin 632. As the primary trigger 152 is rotated about the pivot point formed from the trigger pin 626, the yoke 614 translates horizontally. A spring 634 is used to bias the yoke 614 forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the transducer 104 to engage the ultrasonic mode of the surgical instrument 102.

The elongated shaft assembly 112 includes an outer tubular sheath 660 and a rotation knob 662 coupled to the outer tubular sheath 660. The rotation knob 662 is operable to rotate the outer tubular sheath 660 about an axis defined by the outer tubular sheath 660. A reciprocating tubular actuator 654 is located within the outer tubular sheath 660 and mechanically engaged with the end effector 120 on a distal end. The reciprocating tubular actuator 654 is also mechanically engaged, on a proximal end, with the yoke 614 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly, or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 660, the reciprocating tubular actuator 654, and the ultrasonic waveguide 670 are mechanically engaged together via a pin 658.

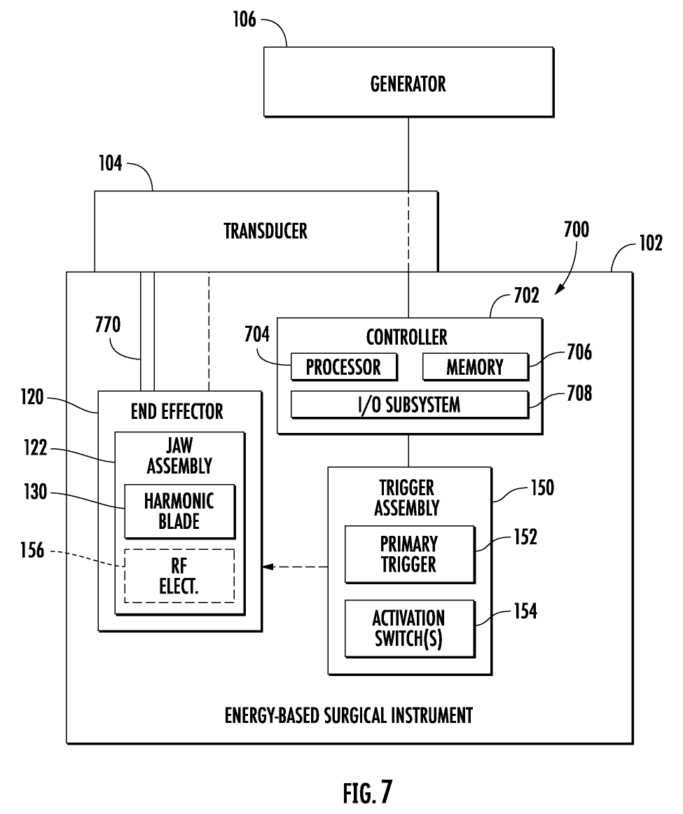

Referring now to FIG. 7, in the illustrative embodiment, the surgical instrument 102 includes a control circuit 700. The control circuit 700 includes a controller 702 and the trigger assembly 150, which cooperate to provide ultrasonic energy to the ultrasonic blade 130 of the jaw assembly 122 of the end effector 120 and/or RF energy to the RF electrodes 156 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or the switch assembly 154 of the trigger assembly 150. In response to activation of the appropriate trigger 152 or switch 154, the controller 702 controls the transducer 104 to generate the ultrasonic energy, which is propagated to the ultrasonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 156 via interconnections 770. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

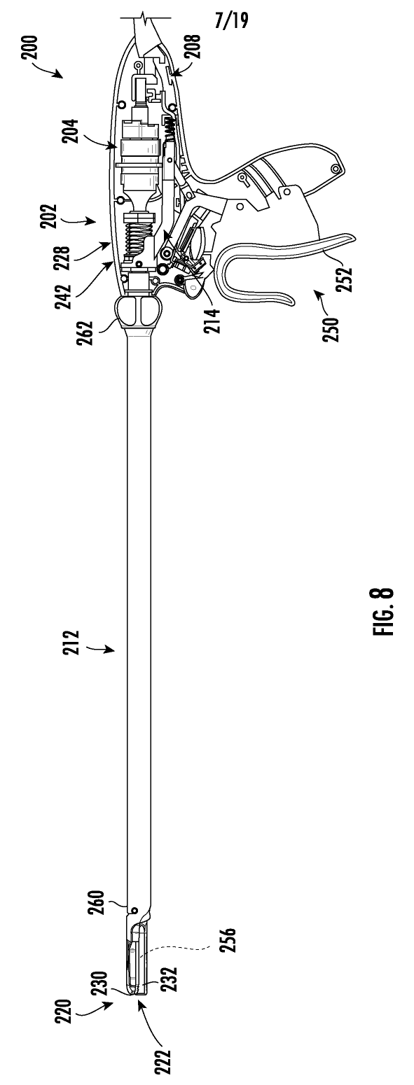

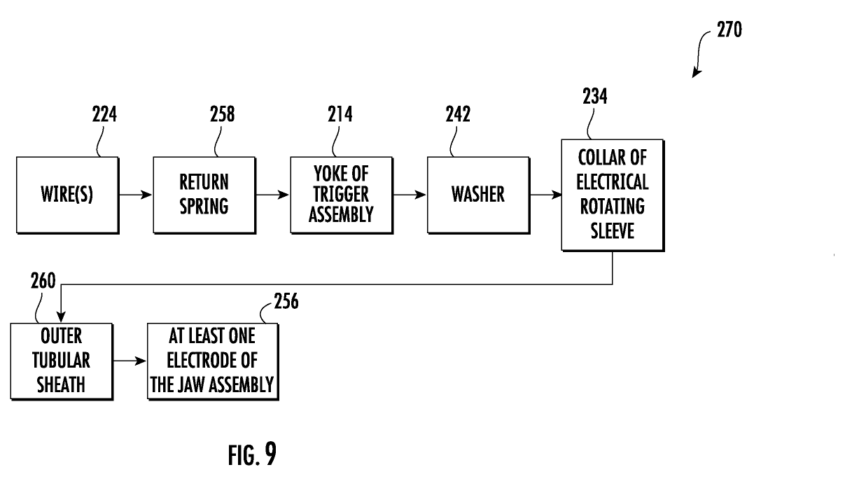

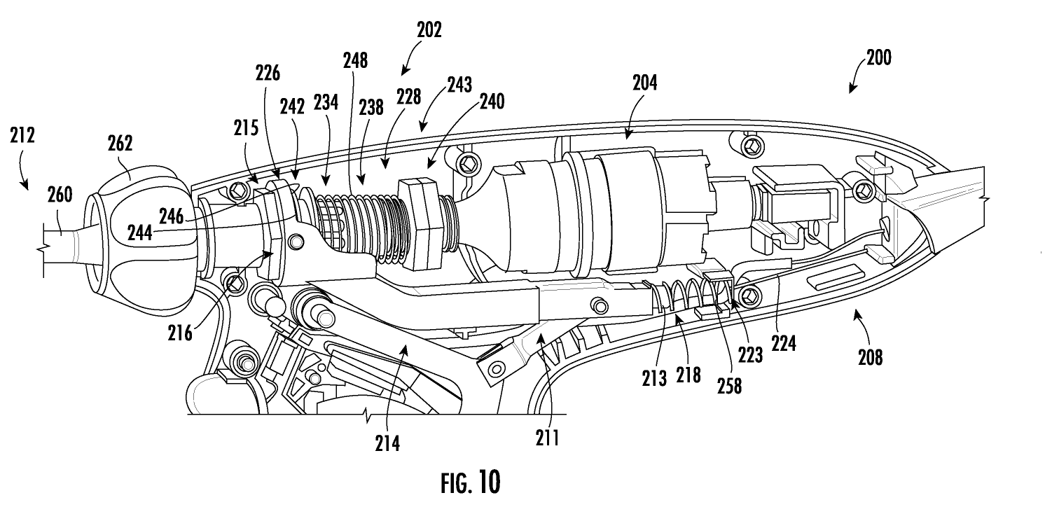

Referring now to FIG. 8 and as discussed further below, in another embodiment, a surgical instrument 202 includes an electrical connection 270 (see FIG. 9) that is capable of rotation or movement during operation of the surgical instrument 202 to transfer an amount of bipolar energy through a trigger assembly 250 to an electrode(s) 256 located in an end effector 220 of the surgical instrument 202. The surgical instrument 202 of FIG. 8 is substantially similar to the surgical system 100 shown in FIGS. 1-7 and described herein. Accordingly, similar reference numbers are used in the description of the surgical instrument 202 to indicate features that are common between the surgical instrument 102 and the surgical instrument 202. The description of the surgical instrument 102 is incorporated by reference to apply to the surgical system 202, except in instances when it conflicts with the specific description and the drawings of the surgical instrument 202.

As shown in FIG. 8, the surgical instrument 202 for performing an energy-based surgical procedure includes a transducer 204 and an electrical rotating sleeve 228 for delivering bipolar energy to a jaw clamp 232. The electrical rotating sleeve 228 can deliver the RF Bipolar energy to the jaw clamp 232 via an outer tubular sheath 260 while minimizing rotational friction between components. In this way, the electrical rotating sleeve 228 allows for the delivery of the RF bipolar energy through the trigger assembly 250 to the electrode(s) 256 of the jaw clamp 232 while allowing the outer tubular sheath 260 to rotate in response to rotation of a rotational knob 262. The electrical rotating sleeve 228 may also be referred to as a “rotating electrical contact.”

The illustrative surgical instrument 202 includes an elongated shaft assembly 212, the trigger assembly 250, a washer 242, and the electrical rotating sleeve 228. The elongated shaft assembly 212 includes the outer tubular sheath 260 and an end effector 220 positioned at a distal end of the outer tubular sheath 260. The end effector 220 includes the jaw assembly 222 that is movable between an open state and a closed state. The jaw assembly 222 includes at least one electrode 256. In some embodiments, the jaw assembly 222 includes an ultrasonic blade 230.

The trigger assembly 250 includes a primary trigger 252 and a yoke 214, as shown in FIG. 8. The yoke 214 is configured to move along a yoke axis in response to operation of the primary trigger 252 to move the jaw assembly 222 between the open state and the closed state. In some embodiments, the yoke 214 is formed of an electrically conductive material. Additionally, in some embodiments, the yoke 214 is formed via machining, metal injection molding (MIM), stamping, three-dimensional (3D) printing, or any other suitable process.

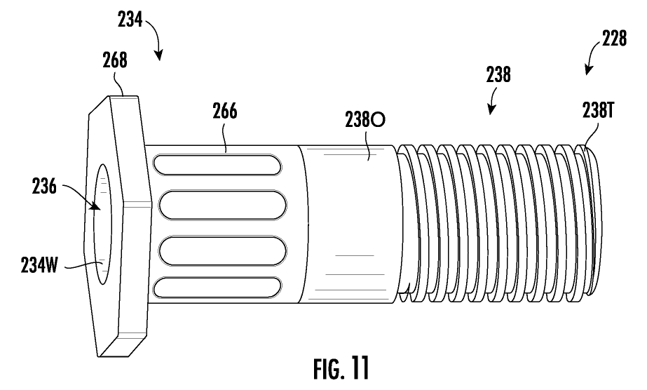

As shown best in FIG. 10, the yoke 214 includes a longitudinal arm 211 that extends between a spring contact 213 at a proximal end 218 thereof and a receiving contact 215 at a distal end 226 thereof. The spring contact 213 at the proximal end 218 of the yoke 214 is coupled to a return spring 258. The return spring 258 extends between the spring contact 213 at the proximal end 218 of the yoke 214 and a plate 223. The return spring 258 is configured to bias the yoke 214 distally such that the jaw assembly 222 of the end effector 220 is biased to the open state. In illustrative embodiments, the plate 223 is formed of an electrically conductive material. The plate 223 is coupled to a wire 224 of a cable assembly 208, as shown in FIG. 10. In some embodiments, the wire 224 is connected to the plate 223 via soldering, crimping, or any other suitable method. A generator, such as the generator 106, can apply RF Bipolar energy to the jaw assembly 122 via the wire 224 of the cable assembly 208. Additionally, as shown best in FIG. 10, the yoke 214 defines a through hole 216 at the distal end 226 thereof. The outer tubular sheath 260 extends through the through hole 216.

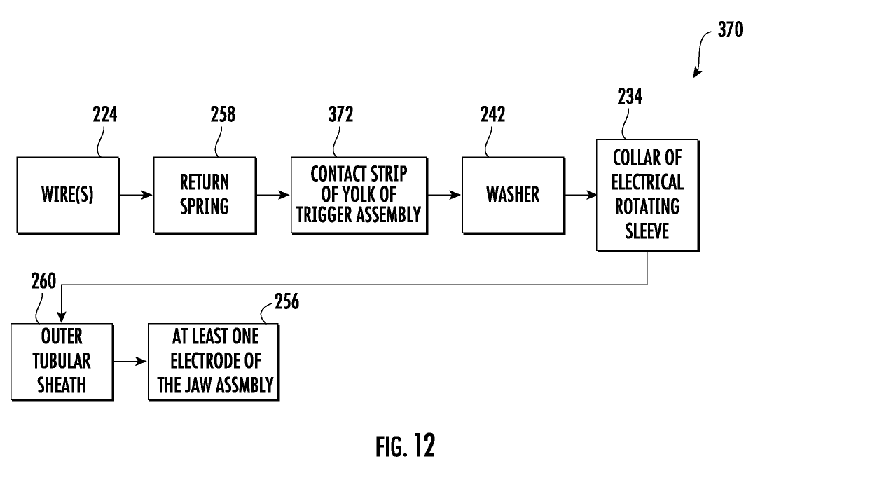

The electrical rotating sleeve 228 is configured to deliver RF bipolar energy to the at least one electrode 256 of the jaw assembly 222 via the outer tubular sheath 260. The electrical rotating sleeve 228 includes a collar 234 and a cylindrical overmold 238 coupled to the collar 234, as shown in FIG. 11. The collar 234 is located between the receiving contact 215 of the yoke 214 and the cylindrical overmold 238.

The collar 234 has an internal wall 234W that defines a bore 236, as best shown in FIG. 11. The bore 236 receives a proximal end of the outer tubular sheath 260 therein, as best shown in FIG. 10. The electrical rotating sleeve 228 can increase the conductive surface area on the outer tubular sheath 260 by receiving at least a portion of the outer tubular sheath 260 therein. In illustrative embodiments, the collar 234 and the outer tubular sheath 260 are fixed to one another. In some embodiments, the collar 234 and the outer tubular sheath 260 are coupled via a cold working process, such as swaging or crimping, welding, adhesives, conductive adhesives, or any other suitable process. In this way, the electrical rotating sleeve 228 rotates with the outer tubular sheath 260 in response to rotation of the rotational knob 262.

In some embodiments, the collar 234 is formed of an electrically conductive material. The collar 234 includes a base 266 and a flange 268 extending outwardly away from a distal edge of the base 266. The base 266 extends through the through hole 216 of the yoke 214 such that the flange 268 is located on a distal side of the yoke 214 (i.e., on a distal side of the receiving contact 215 of the yoke 214). The collar 234 may also be referred to as a flanged bushing.

The cylindrical overmold 238 is coupled between the collar 234 and the transducer 204, as shown best in FIG. 10. In illustrative embodiments, the cylindrical overmold 238 is formed of a non-electrically conductive material. As shown in FIG. 11, the cylindrical overmold 238 includes a set of external threads 238T formed on an outer surface 238O thereof. In some embodiments, the set of external threads 238T extends only along a portion of the cylindrical overmold 238. In some embodiments, the plurality of external threads 238T extends along an entirety of the cylindrical overmold 238. As shown in FIG. 10, a nut 240 is received on the cylindrical overmold 238 in engagement with the set of external threads 238T. The set of external threads 238T allows for translating of one or more components, such as the nut 240, along the cylindrical overmold 238.

As shown in FIG. 10, a washer 242 is received on the electrical rotating sleeve 228. The washer 242 may also be referred to as a distal washer 242 of a spring stack assembly 243. The washer 242 extends around the collar 234 of the electrical rotating sleeve 228 such that the washer 242 is in direct contact with the collar 234. In illustrative embodiments, the washer 242 is formed of an electrically conductive material. In this way, direct contact between the washer 242 and the collar 234 provides electrical contact between the washer 242 and the outer tubular sheath 260 through the collar 234.

A distal face 244 of the washer 242 is in direct contact with a proximal face 246 of the receiving contact 215 of the yoke 214, as shown in FIG. 10. A portion of the yoke 214 is, thus, located between the flange 268 and the washer 242. A spring 248 of the spring stack assembly 243 extends between the washer 242 and the nut 240 to maintain the direct contact of the washer 242 with the proximal face 246 of the yoke 214. The direct contact between the yoke 214 and the washer 242 establishes electrical contact between the yoke 214 and the washer 242, and thus, the collar 234 and the outer tubular sheath 260.

An electrical connection 270 is established through the surgical instrument 202 to provide the RF Bipolar energy through the trigger assembly 250 to the electrode(s) 256 while allowing the outer tubular sheath 260 to rotate in response to rotation of the rotational knob 262. For example, as shown in FIG. 9, the electrical connection 270 is established between the wire 224 of the cable assembly 208, to the return spring 258 (via the plate 223), and to the yoke 214. The electrical connection 270 is then established from the yoke 214, to the washer 242, to the collar 234 of the electrical rotating sleeve 228, to the outer tubular sheath 260, and to the electrode(s) 256. The wire 224, the plate 223, the return spring 258, the yoke 214, the washer 242, the collar 234, and the outer tubular sheath 260 all comprise an electrically conductive material to allow for the formation of the electrical connection 270. In this way, the RF bipolar energy can be delivered from the cable assembly 208 to the target tissue. The direct contact and connection between the collar 234 and the proximal end of the outer tubular sheath 260 provides for an increased conductive surface area on the outer tubular sheath 260. The electrical rotating sleeve 228 allows for electrical connection between the non-rotatable yoke 214 and the non-rotatable washer 242 with the rotatable outer tubular sheath 260.

Referring now to FIGS. 13-15, in another embodiment, a surgical instrument 302 includes a trigger assembly 350 having a yoke 314 configured to provide the electrical connection 370 through the surgical instrument 302. The trigger assembly 350 includes the primary trigger 252 and the yoke 314, as shown in FIG. 13. The yoke 314 is configured to move along a yoke axis in response to operation of the primary trigger 252 to move the jaw assembly 222 between the open state and the closed state. The yoke 314 includes a support arm 311 and a contact strip 372 that moves with the support arm 311 along the yoke axis, as shown in FIGS. 13-15. The contact strip 372 extends along the support arm 311. The contact strip 372 can be free or attached to the support arm 311 for assembly feasibility.

In the illustrative embodiment, the contact strip 372 is formed of an electrically conductive material. Additionally, in the illustrative embodiment the support arm 311 is formed of a non-electrically conductive material. The support arm 311 extends between a boss 374 at a proximal end 318 thereof and a receiving hoop 315 at a distal end 326 thereof, as shown in FIG. 15. As shown in FIG. 13, the boss 374 at the proximal end 318 of the support arm 311 is coupled to the return spring 258. The receiving hoop 315 at the distal end 326 of the support arm 311 defines a through hole 316, and the outer tubular sheath 260 extends through the through hole 316.

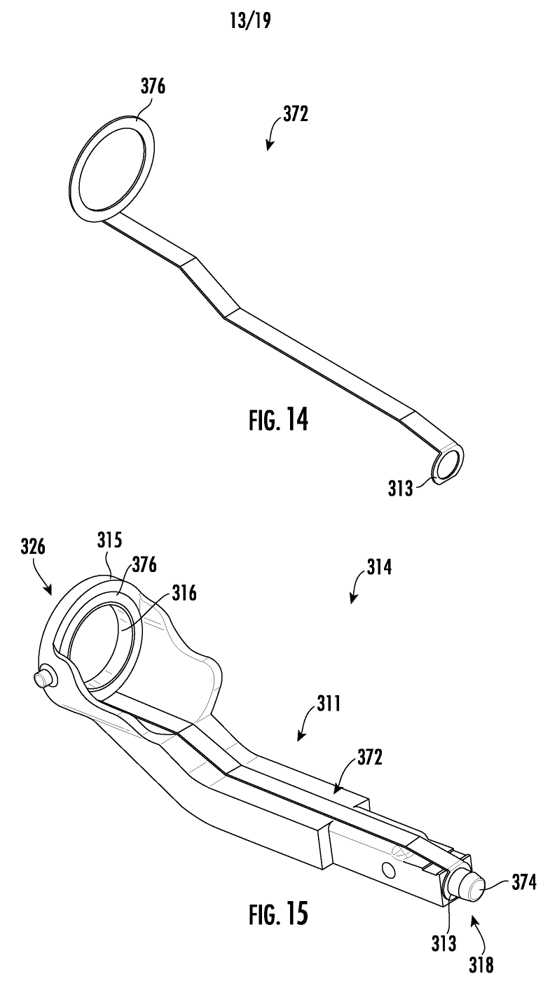

The contact strip 372 extends between a spring contact 313 at a proximal end thereof and a receiving contact 376 at a distal end thereof, as shown in FIG. 14. The spring contact 313 is received on the boss 374 of the support arm 311, and the spring contact 313 contacts the return spring 258, as shown in FIG. 13. T he receiving contact 376 extends around the through hole 316 of the receiving hoop 315 of the support arm 311 to receive the outer tubular sheath 260 therethrough such that the outer tubular sheath 260 extends into the electrical rotating sleeve 228.

The distal face 244 of the washer 242 is in direct contact with the receiving contact 376 of the contact strip 372, as shown in FIG. 13. The direct contact between the contact strip 372 and the washer 242 establishes electrical contact between the contact strip 372 and the washer 242, and thus, the collar 234 and the outer tubular sheath 260.

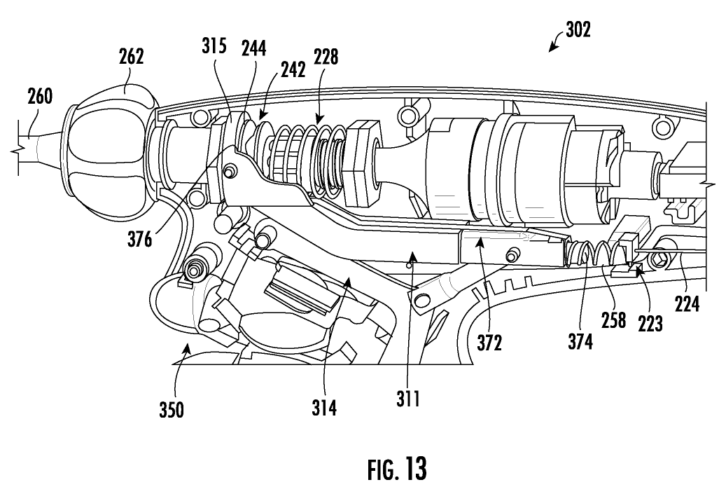

The electrical connection 370 is established through the surgical instrument 202 to provide the RF bipolar energy through the trigger assembly 350 to the electrode(s) 256 while allowing the outer tubular sheath 260 to rotate in response to rotation of the rotational knob 262. As shown in FIG. 12, the electrical connection 370 is established between the wire 224 of the cable assembly 208, to the return spring 258 (via the plate 223), and to the contact strip 372 of the yoke 314. The electrical connection 370 is then established from the contact strip 372 of the yoke 314, to the washer 242, to the collar 234 of the electrical rotating sleeve 228, to the outer tubular sheath 260, and to the electrode(s) 256. The wire 224, the plate 223, the return spring 258, the contact strip 372 of the yoke 314, the washer 242, the collar 234, and the outer tubular sheath 260 all comprise an electrically conductive material to allow for the formation of the electrical connection 370.

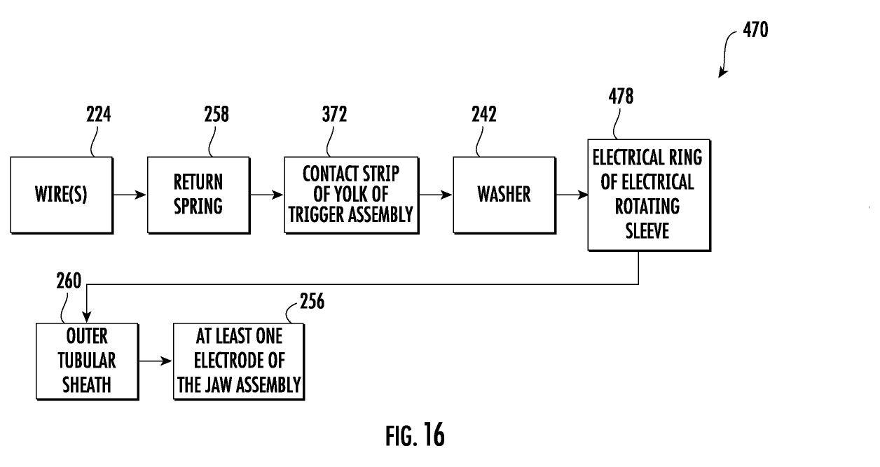

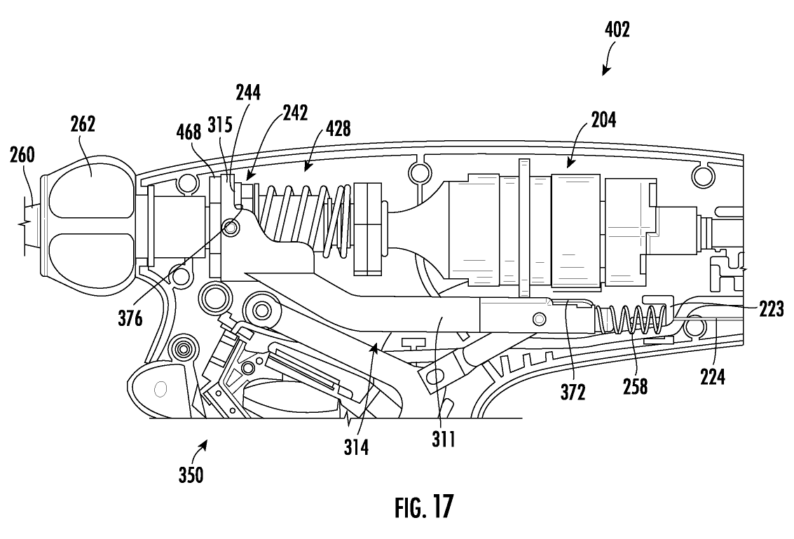

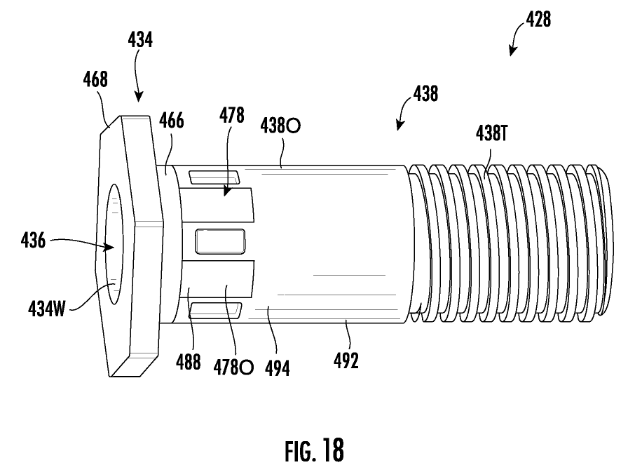

Referring now to FIGS. 17-21, in another embodiment, a surgical instrument 402 for performing an energy-based surgical procedure includes the trigger assembly 350 and an electrical rotating sleeve 428. The electrical rotating sleeve 428 provides an alternate electrical connection 470 through the surgical instrument 402, as shown in FIG. 16. The electrical rotating sleeve 428 can deliver the RF bipolar energy via the outer tubular sheath 260 while minimizing rotational friction between components. In this way, the electrical rotating sleeve 428 allows for the delivery of the RF Bipolar energy through the trigger assembly 350 to the electrode(s) 256 while allowing the outer tubular sheath 260 to rotate in response to rotation of the rotational knob 262.

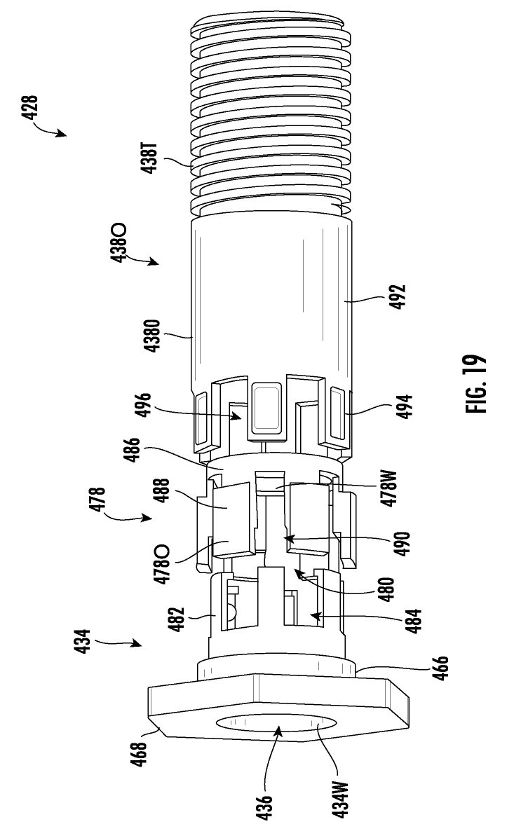

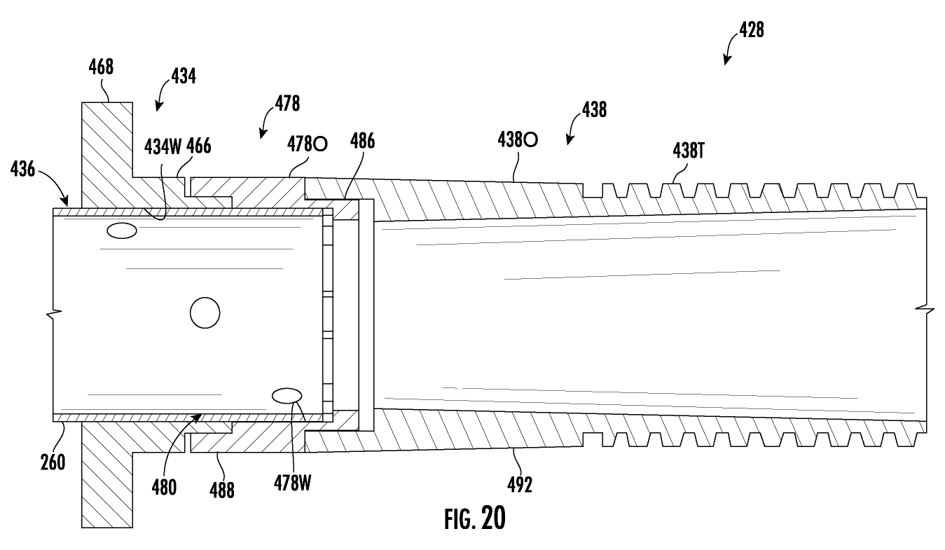

The electrical rotating sleeve 428 includes a collar 434, a cylindrical overmold 438, and an electrical ring 478, as shown in FIGS. 18 and 19. The electrical ring 478 is located between the collar 434 and the cylindrical overmold 438. The collar 434 has an internal wall 434W that defines a bore 436, as shown in FIG. 20. The bore 436 receives a proximal end of the outer tubular sheath 260 therein. The electrical ring 478 also has an internal wall 478W that defines a bore 480 that is co-axial with the bore 436. The proximal end of the outer tubular sheath 260 extends through the bore 436 and into the bore 480, as shown in FIG. 20.

In illustrative embodiments, the collar 434 is formed of a non-electrically conductive material. The collar 434 includes a band 466, a flange 468 extending outwardly away from a distal edge of the band 466, and a plurality of tabs 482. The band 466 extends through the through hole 316 of the receiving hoop 315 of the support arm 311 such that the flange 468 is located on a distal side of the receiving hoop 315 of the yoke 314, as shown in FIG. 17. The plurality of tabs 482 extends proximally away from a proximal edge of the band 466, as shown in FIG. 19. Each of the plurality of tabs 482 is circumferentially spaced apart from one another about the band 466. The plurality of tabs 482 cooperates to define a tab channel 484 between each pair of adjacent tabs 482 of the plurality of tabs 482.

The electrical ring 478 is disposed between the cylindrical overmold 438 and the collar 434, as shown in FIGS. 18 and 19. In illustrative embodiments, the electrical ring 478 is formed of an electrically conductive material. The electrical ring 478 includes a base 486 and a set of fingers 488 extending distally away from the base 486 toward the collar 434. Each of the set of fingers 488 is circumferentially spaced apart from one another about the base 486. The set of fingers 488 cooperates to define a finger channel 490 between each pair of adjacent fingers 488 of the plurality of fingers 488. Each of the set of fingers 488 of the electrical ring 478 is received in a corresponding tab channel 484 of the collar 434 between adjacent tabs 482, as suggested in FIG. 19. As shown in FIG. 20, a distal end of each of the set of fingers 488 extends over a proximal end of the band 466 of the collar 434. E ach of the set of tabs 482 of the collar 434 is received in a corresponding finger channel 490 of the electrical ring 478 between adjacent fingers 488.

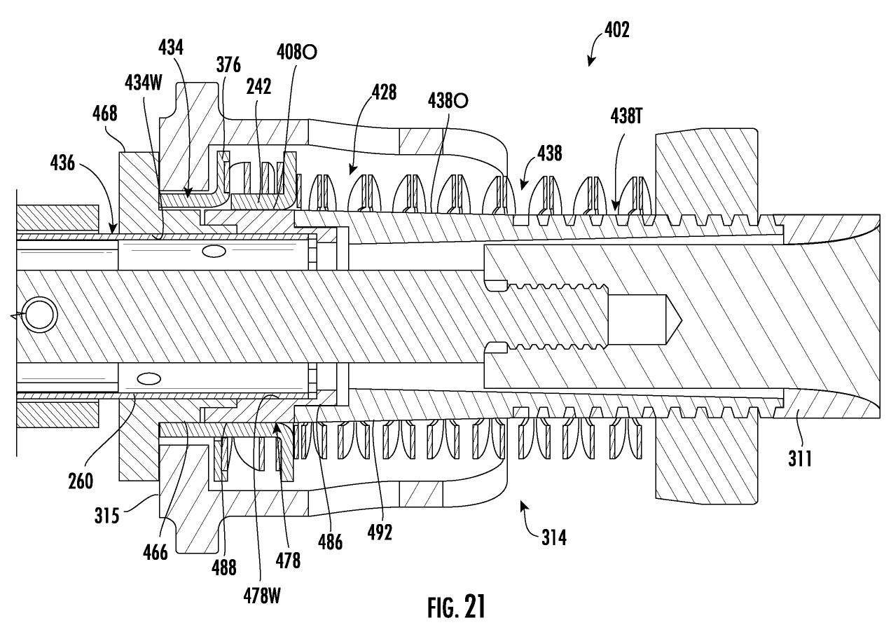

As shown in FIGS. 20 and 21, the internal wall 478W of the electrical ring 478 directly contacts the outer tubular sheath 260. As shown in FIG. 21, the washer 242 is received on the electrical rotating sleeve 428. The washer 242 extends around the electrical ring 478 of the electrical rotating sleeve 428 such that the washer 242 is in direct contact with an outer wall 478O of the electrical ring 478. In this way, direct contact between the washer 242 and the electrical ring 478 provides electrical contact between the washer 242 and the outer tubular sheath 260 through the electrical ring 478.

The distal face 244 of the washer 242 is in direct contact with the receiving contact 376 of the contact strip 372, as shown in FIGS. 17 and 21. The direct contact between the contact strip 372 and the washer 242 establishes electrical contact between the contact strip 372 and the washer 242, and thus, the electrical ring 478 and the outer tubular sheath 260.

The cylindrical overmold 438 is coupled between the electrical ring 478 and the transducer 204. In illustrative embodiments, the cylindrical overmold 438 is formed of a non-electrically conductive material. As shown in FIG. 18, the cylindrical overmold 438 includes a set of external threads 438T formed on an outer surface 438O thereof. In some embodiments, the set of external threads 438T extends only along a portion of the cylindrical overmold 438. In some embodiments, the set of external threads 438T extends along an entirety of the cylindrical overmold 438.

The cylindrical overmold 438 includes a body 492 and a set of extensions 494 extending distally away from the body 492, as shown in FIGS. 18 and 19. Each of the set of extensions 494 is circumferentially spaced apart from one another about the body 492. The set of extensions 494 cooperates to define an extension channel 496 between each pair of adjacent extensions 494 of the plurality of extensions 494. Each of the plurality of extensions 494 of the cylindrical overmold 438 is received in a corresponding finger channel 490 of the electrical ring 478 between adjacent fingers 488. In other words, each of the plurality of fingers 488 of the electrical ring 478 is received in a corresponding extension channel 496 of the cylindrical overmold 438 between adjacent extensions 494.

A distal portion of the body 492 overlaps with the base 486 of the electrical ring 478, as shown in FIG. 20. Each of the set of extensions 494 overlaps with a corresponding one of the set of tabs 482 of the collar 434, as suggested in FIGS. 18 and 19. In this way, each of the set of extensions 494 is in contact with a corresponding one of the set of tabs 482. Each of the set of extensions 494 is coupled and/or fixed to a corresponding one of the set of tabs 482 thereby trapping the electrical ring 478 between the collar 434 and the cylindrical overmold 438. The collar 434 and the cylindrical overmold 438 may be coupled and/or fixed to one another via plastic laser welding, adhesives, press-fit connections, or any other suitable method.

An electrical connection 470 is established through the surgical instrument 402 to provide the RF bipolar energy through the trigger assembly 350 to the electrode(s) 256 while allowing the outer tubular sheath 260 to rotate in response to rotation of the rotational knob 262. As shown in FIG. 16, the electrical connection 470 is established between the wire 224 of the cable assembly 208, to the return spring 258 (via the plate 233), and to the contact strip 372 of the yoke 314. The electrical connection 470 is then established from the contact strip 372 of the yoke 314, to the washer 242, to the electrical ring 478 of the electrical rotating sleeve 428, to the outer tubular sheath 260, and to the electrode(s) 256. The wire 224, the plate 223, the return spring 258, the contact strip 372 of the yoke 314, the washer 242, the electrical ring 478, and the outer tubular sheath 260 may all be formed from an electrically conductive material to allow for the formation of the electrical connection 470. The RF bipolar energy can be delivered from the cable assembly 208 to the target tissue.

The direct contact and connection between the electrical ring 478 and the proximal end of the outer tubular sheath 260 provides for an increased conductive surface area on the outer tubular sheath 260. The size, length, and number of fingers 488, tabs 482, and/or extensions 494 on each of the components can be varied to provide additional surface area for attachment or electrical connection redundancy. In some embodiments, RF energy may be transmitted to the one or more RF electrodes 256 incorporated into the jaw clamp 232 of the end effector 220 while allowing for relative motion between components. For example, in some embodiments, the elongated shaft assembly 212 of the present embodiments can move axially and/or rotationally while RF energy is transmitted to the jaw clamp 232. Additionally, it will be appreciated that while the figures illustrate the surgical instrument 402 with the yoke 314, the surgical instrument 402 of the present embodiment can be used with the yoke 214 of FIG. 10 in some embodiments.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A surgical instrument comprising:
an elongated shaft assembly having an outer tubular sheath and an end effector positioned at a distal end of the outer tubular sheath, the end effector having a jaw assembly movable between an open state and a closed state, wherein the jaw assembly includes at least one electrode and an ultrasonic blade;
a trigger assembly operable to move the jaw assembly of the end effector between the open state and the closed state;
a washer in contact with the trigger assembly; and
an electrical rotating sleeve for delivering bipolar energy to the at least one electrode of the jaw assembly via the outer tubular sheath, wherein the washer is received on the electrical rotating sleeve and a proximal end of the outer tubular sheath is received within the electrical rotating sleeve, and wherein an electrical connection is established between the trigger assembly, the washer, the electrical rotating sleeve, and the outer tubular sheath to deliver the bipolar energy to the at least one electrode.

2. The surgical instrument of claim 1, wherein the electrical rotating sleeve includes a collar having an internal wall that defines a bore in which the outer tubular sheath is received and a cylindrical overmold coupled to the collar, wherein the cylindrical overmold includes a plurality of external threads formed on an outer surface thereof.

3. The surgical instrument of claim 2, wherein the washer extends around the collar of the electrical rotating sleeve to provide electrical contact between the trigger assembly and the outer tubular sheath via the collar.

4. The surgical instrument of claim 3, wherein the trigger assembly includes a primary trigger and a yoke configured to move along a yoke axis in response to operation of the primary trigger to move the jaw assembly of the end effector between the open state and the closed state, and wherein the washer is in contact with the yoke of the trigger assembly.

5. The surgical instrument of claim 4, wherein the yoke of the trigger assembly and the collar of the electrical rotating sleeve are formed of an electrically conductive material.

6. The surgical instrument of claim 4, wherein the yoke includes a support arm and a contact strip, wherein the contact strip extends along the support arm and is in electrical contact with the washer.

7. The surgical instrument of claim 6, wherein the contact strip of the yoke and the collar of the electrical rotating sleeve are formed of electrically conductive materials, and wherein the support arm of the yoke is formed of non-electrically conductive materials.

8. The surgical instrument of claim 2, wherein the electrical rotating sleeve further includes an electrical ring disposed between the collar and the cylindrical overmold, the electrical ring having a base and a plurality of fingers extending distally away from the base toward the collar, the plurality of fingers cooperating to define a finger channel between each pair of adjacent fingers of the plurality of fingers, and wherein the washer extends around and is in electrical contact with the electrical ring of the electrical rotating sleeve to provide electrical contact between the trigger assembly and the outer tubular sheath via the electrical ring.

9. The surgical instrument of claim 8, wherein the collar of the electrical rotating sleeve includes a band, a flange extending outwardly away from the band at a distal end thereof, and a plurality of tabs extending proximally away from a proximal end of the band toward the electrical ring, and wherein the plurality of tabs cooperates to define a tab channel between each pair of adjacent tabs of the plurality of tabs.

10. The surgical instrument of claim 9, wherein each of the plurality of fingers of the electrical ring is received in a corresponding tab channel of the plurality of tab channels, and wherein each of the plurality of tabs of the band is received in a corresponding finger channel of the plurality of finger channels.

11. The surgical instrument of claim 10, wherein the cylindrical overmold includes a body and a plurality of extensions extending distally away from the body into the plurality of finger channels of the electrical ring and into contact with the plurality of tabs of the collar.

12. A surgical instrument comprising:
an elongated shaft assembly having an outer tubular sheath and an end effector positioned at a distal end of the outer tubular sheath, the end effector having a jaw assembly movable between an open state and a closed state, wherein the jaw assembly includes at least one electrode;
a trigger assembly operable to move the jaw assembly of the end effector between the open state and the closed state; and
an electrical rotating sleeve for delivering bipolar energy to the at least one electrode of the jaw assembly through the outer tubular sheath, wherein the electrical rotating sleeve is configured to receive a proximal end of the outer tubular sheath therein to rotate therewith, wherein the bipolar energy is directed through the trigger assembly, to the electrical rotating sleeve, through the outer tubular sheath, and to the at least one electrode.

13. The surgical instrument of claim 12, wherein the electrical rotating sleeve includes a collar having an internal wall that defines a bore in which the outer tubular sheath is received and a cylindrical overmold coupled to the collar, wherein the cylindrical overmold includes a plurality of external threads formed on an outer surface thereof.

14. The surgical instrument of claim 13, further comprising a washer that extends around the collar of the electrical rotating sleeve to provide electrical contact between the trigger assembly and the outer tubular sheath via the collar.

15. The surgical instrument of claim 13, wherein the electrical rotating sleeve further includes an electrical ring disposed between the collar and the cylindrical overmold, the electrical ring having a base and a plurality of fingers extending distally away from the base toward the collar, and wherein the surgical instrument further includes a washer that extends around and is in electrical contact with the electrical ring to provide electrical contact between the trigger assembly and the outer tubular sheath via the electrical ring.

16. The surgical instrument of claim 15, wherein the collar includes a band, a flange extending outwardly away from the band at a distal end thereof, and a plurality of tabs extending proximally away from a proximal end of the band toward the electrical ring, and wherein each of the plurality of fingers of the electrical ring is received between adjacent tabs of the plurality of tabs of the collar.

17. The surgical instrument of claim 16, wherein the cylindrical overmold includes a body and a plurality of extensions extending distally away from the body, each of the plurality of extensions of the cylindrical overmold being received between adjacent fingers of the plurality of fingers of the electrical ring, and wherein each of the plurality of extensions extends over a corresponding tab of the plurality of tabs of the collar.

18. A method comprising:
positioning a proximal end of an outer tubular sheath of an elongated shaft assembly in an electrical rotating sleeve to provide electrical contact between the outer tubular sheath and the electrical rotating sleeve;
positioning a washer around the electrical rotating sleeve to provide electrical contact between the washer and the electrical rotating sleeve;
contacting the washer with a yoke of a trigger assembly to provide electrical contact between the yoke and the washer; and
directing bipolar energy through the yoke, to the washer, to the electrical rotating sleeve, through the outer tubular sheath, and to at least one electrode of a jaw assembly included in the elongated shaft assembly.

19. The method of claim 18, wherein the electrical rotating sleeve includes a collar, a cylindrical overmold, and an electrical ring disposed between the collar and the cylindrical overmold, the collar and the cylindrical overmold being formed of non-electrically conductive materials, and
wherein positioning the proximal end of the outer tubular sheath of the elongated shaft assembly in the electrical rotating sleeve to provide electrical contact between the outer tubular sheath and the electrical rotating sleeve includes contacting the electrical ring of the electrical rotating sleeve with the proximal end of the outer tubular sheath.

20. The method of claim 19, wherein positioning the washer around the electrical rotating sleeve to provide electrical contact between the washer and the electrical rotating sleeve includes contacting the electrical ring of the electrical rotating sleeve with the washer.
